(19) United States
(12) Patent Application Publication
(10) Pub. No.: US 2015/0299238 A1
(43) Pub. Date: Oct. 22, 2015

(54) SYNTHESIS AND USE OF PRODRUG COMPLEXES OF COBALT IN POLYMER THERAPEUTICS

(71) Applicant: The University of North Texas, Denton, TX (US)

(72) Inventors: Robby A. Petros, Denton, TX (US); Duong T. Nguyen, Denton, TX (US); Ronaldo J. Cavazos, Denton, TX (US); Clifford S. Morrison, Denton, TX (US); Jana B. Lampe, Denton, TX (US); Alesha N. Harris, Denton, TX (US)

(21) Appl. No.: 14/692,599

(22) Filed: Apr. 21, 2015

Related U.S. Application Data
(60) Provisional application No. 61/982,667, filed on Apr. 22, 2014.

(51) Int. Cl. C07F 15/06; A61K 9/51; A61K 31/4745

(52) U.S. Cl. CPC C07F 15/06; A61K 31/4745; A61K 9/5169

(57) ABSTRACT

Degradable compounds that can be controlled to degrade and can be used for delivery of a cargo component. Cobalt (III) complexes have been exploited as vehicles for molecular complexes. This disclosure describes the use of such complexes as bioconjugation reagents in crosslinking proteins to form particles, PEGylation of proteins, and the synthesis of (bio)polymer-drug conjugates. The Co-based linkages can be designed to respond to internal stimuli, such as changes in pH and reduction potential, or external stimuli, such as applied electromagnetic radiation. Therapeutics are entrapped within these crosslinked particles, covalently attached to the polymer making up the matrix through additional stimuli-responsive linkages, or could comprise the matrix itself.

This application claims priority to U.S. Provisional Patent Application Ser. No. 61/982,667, entitled “Synthesis and Use of Prodrug Complexes of Cobalt in Polymer Therapeutics,” filed on Apr. 22, 2014, the entire contents of which are hereby incorporated by reference.

### BACKGROUND

One aspect of the present disclosure is directed to degradable compounds that may be used for target delivery of a cargo component. More particularly, the degradable compounds may comprise a composition that degrades under specified conditions and that may release a component contained in the composition.

Devices and methods for delivery of desired components to a site of interest remain a growing need. For example, a variety of methods and routes of administration have been developed to deliver pharmaceuticals, such as small molecular drugs and other biologically active compounds (e.g., peptides, hormones, proteins, and enzymes). Many routes of administration are known for delivering desired pharmaceuticals to a patient. As greater knowledge is learned regarding toxicity of drugs and the ability to elicit specific responses by delivery of a pharmaceutical only to a specific portion of the body, controlled release of pharmaceuticals after their administration has become highly important.

Gene therapy, for example, is a promising field; however, such therapy requires gene or polynucleotide transfer across the cell membrane and into the nucleus where the gene can be expressed. Many conventional drug delivery techniques simply cannot provide a delivery vehicle of sub-cellular dimensions that can effectively deliver specific materials to individual cells. The effectiveness of drugs and other compounds can also be increased by target specific delivery, and the use of micro- or nano-sized delivery devices can be particularly beneficial to increase drug activity while actually reducing the overall concentration of drug delivered. Accordingly, there is a need in the art for compounds and methods useful to facilitate delivery and release of desired compounds to a site of interest, particularly on a micro- or nano-size scale.

### SUMMARY

One aspect of the present disclosure is directed to compounds that will degrade under specified conditions, methods of using such compounds in target drug delivery, and compositions comprising such compounds. The degradable compounds may be characterized by the labile —Co-A- groups present in the compounds (A representing an atom, such as O, N, or S, or a group, such as C═O). The compounds are stable under defined conditions but are degradable under specified conditions, such as, for example, physiological temperature (e.g., about 37 degrees C), acidic, or reducing conditions. The compounds may be incorporated into a composition including a polymeric matrix and/or a cargo component. In certain embodiments, the polymeric matrix can be any material useful for forming discrete particles. Of course, depending upon the desired mode of delivery, the matrix material can comprise any number of materials useful for physically or chemically combining with the cargo component. In some embodiments, the degradable compound may be used as a crosslinker material, thus forming a part of the matrix material. In other embodiments, the matrix material may be substantially completely formed of the degradable compound. A wide variety of cargo components also may be used in the present invention. In particular embodiments, the cargo component comprises a drug or other therapeutic agent. Accordingly, the disclosure particularly provides methods of target delivery of a drug or other therapeutic material. In one aspect, the invention is particularly directed to degradable compounds whose degradation can be controlled. According to certain embodiments, a degradable compound according to one aspect may have a structure according to Formula (Ia),

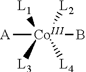

wherein: A and B are the same or different and contain NH₃, NH₂R, NHR₁R₂, NR₁R₂R₃, OH₂, OHR, OHR₁R₂, SH₂, SHR₁, SHR₂, PH₃, PH₂R₁, PHR₁R₂, PR₁R₂R₃; R₁, R₂, and R₃ are the same or different and are optionally substituted straight or branched chain C₁-C₆ alkyl, alkenyl, or alkynyl; optionally substituted C₃-C₆ cycloalkyl, cycloalkenyl, cycloalkynyl, or hydrogen; optionally substituted phenyl or benzyl; or optionally substituted polyether or polyester; Co may contain up to four other bound ligands (L₁-L₄) including H, X (X being F, Cl, Br, or I), OR₂, NR₃, OR, SR, NR₂, PR₂, ER₃, EX₃ (E being Si, Ge, or Sn), CH₃ (or other alkyl group), η¹ to η⁷-aryl, η¹ to η⁷-alkenyl, η¹ to η²-alkynyl, η¹ to η²-acyl, η²-ketone, terminal carbene, terminal carbine, CO, CNR, N₂, NO, N₂R, NR, PR₃, PX₃, AsR₃, SbR₃, NR₃, RNCR₂, RCN, ether, thioether, N, O, or O₂.

One aspect of the present disclosure provides compositions comprising the degradable compounds. In certain embodiments, the compositions particularly may comprise a matrix material and a cargo component, which may be a drug, a therapeutic material, or any other compound capable of being physically or chemically combined with the matrix material. The matrix material may be at least partially formed using the degradable compounds, or the degradable material may be otherwise associated with the matrix material (e.g., attached to a surface of a particle formed from the matrix material). Thus, the composition provides for delivery and release of the cargo component through degradation of the composition.

In one embodiment, the composition comprises a cargo component and a matrix material, the matrix material comprising a degradable compound having the structure of Formula (Ia), as described above. In specific embodiments, the degradable compound may comprise about 0.1% to about 50% by weight of the matrix material, about 50% to about 99.9% by weight of the matrix material, or about 10% to about 90% by weight of the matrix material.

In some embodiments, the matrix material may be a co-polymer comprising the degradable compound and one or more co-monomers. In further embodiments, the matrix material may comprise one or more polymers cross-linked with the degradable compound. The composition also may comprise further components, such as a polymerization initiator (e.g., a photoinitiator). In other embodiments, the matrix material specifically may comprise a biodegradable polymer.

The cargo component may be associated with the matrix material via a variety of means. For example, the cargo component may be encapsulated by the matrix material, the cargo component may be the metal complex used for crosslinking, may be physically blended with the matrix material, and/or the cargo component may be covalently bonded to one or more functional groups present on the matrix material. In one embodiment, the matrix material may be in the form of a particle having the degradable compound covalently bonded to one or more functional groups present on an exposed surface of the particle, and the cargo component may be attached to the particle via the degradable compound.

In certain embodiments, the composition may be provided in the form of discrete particles. Thus, the composition may be formed into discrete particles of micro- and nano-scale dimensions. Such particles may be administered directly to a site where it is desirable for the cargo component to be released. In some embodiments, the discrete particles may be incorporated into pharmaceutical formulations.

Accordingly, certain embodiments include a particle comprising a degradable compound having the structure of Formula (Ia), as described herein. The particle further may comprise a cargo component associated with the particle. Also, the particle may be a surface-activated particle. Specifically, the particle may have an exposed surface with a degradable compound attached thereto and including a reactive group that makes the particle activated in that it is ready to receive a further component (e.g., a cargo component) that will covalently attach to the particle via the degradable compound.

A pharmaceutical formulation according to the disclosure may comprise a pharmaceutically acceptable carrier, a pharmaceutical material, and a degradable compound having the structure of Formula (Ia), as described herein. In particular, the formulation may comprise a matrix material including the degradable compound. Still further, the matrix material may be in the form of a particle. In such embodiments, the pharmaceutical material may be associated with the particle (e.g., attached to an exposed surface of the particle via the degradable compound and/or at least partially encapsulated by the particle). In other embodiments, the composition may comprise a first particle type and a second particle type, each particle type comprising a matrix material and a cargo component, and the matrix material of at least one particle type comprising a degradable compound having the structure of Formula (Ia), as described herein.

In particular embodiments, the composition can vary by altering various components of the particles. For example, the first particle type can be different from the second particle type in one or more of the matrix material and the cargo component. Specifically, the polymeric makeup of the matrix material in each particle type may differ, and/or the cargo component used in each particle type may differ, and/or the degradable compound used in each particle type may differ.

In another aspect, the present disclosure also provides methods of treatment. The wide applicability of the degradable particles described above makes them particularly useful in treating a wide variety of conditions and diseases. Virtually any drug or therapeutic agent may be formed into the inventive, degradable particles. Accordingly, certain embodiments provide methods of treating a patient comprising administering to the patient a composition (particularly in the form of discrete particles). Preferably, the composition comprises a drug or therapeutic agent known to prevent, treat, cure, or ameliorate a disease or condition. Thus, in one embodiment, the method may comprise administering to a patient a composition comprising a pharmaceutical material, and a degradable compound having the structure of Formula (Ia), as described herein.

### BRIEF DESCRIPTION OF THE DRAWINGS

FIG. 1 shows the synthesis of albumin nanoparticles using cobalt (II) chloride.

FIG. 2 shows solutions incubated for 14 days at 37° C. with no stirring under the indicated conditions.

FIG. 3 shows stability of particle solutions over time as measured by DLS (incubated at 37° C. without agitation).

FIG. 4 shows cytotoxicity of co-crosslinked albumin nanoparticles in SNU-5 cells.

FIG. 5 shows particle size analysis of the pegylation (PEG, poly (ethylene glycol)) of transferrin (T_{f}).

FIG. 6 shows Dynamic Light Scattering results for Co-Alb SN-38 NPs.

FIG. 7 shows stability of SN-38 loaded Co-Alb NPs.

FIG. 8 shows representative SEM images of Co-Alb NPs.

FIG. 9 shows Dynamic Light Scattering results for Co-Alb-FITC NPs before and after oxidation of cobalt.

FIG. 10 shows (a) Dot plots of side scatter vs. albumin-FITC fluorescence illustrating the kinetics of uptake of Co-Alb-FITC NPs (100 μg/mL dosing) by SNU-5 cells and (b) percentages of cells displaying no, low, or high uptake of Co-Alb-FITC NPs.

FIG. 11 shows (a) Dot plots of side scatter vs. albumin-FITC fluorescence for SNU-5 cells incubated with various concentrations of EIPA illustrating the inhibitory effect on uptake of Co-Alb-FITC NPs after 2 h incubation and (b) relative macropinocytic uptake of Co-Alb-FITC NPs in the presence of varying amounts of an EIPA (4 h incubation time).

FIG. 12 shows (a) Dot plots of side scatter vs. albumin-FITC fluorescence for SNU-5 cells incubated with free FITC-albumin (100 μg/mL, no NPs) illustrating macropinocytic uptake of the free protein itself, and (b) Dot plots of side scatter vs. albumin-FITC fluorescence for Jurkat cells incubated with Co-Alb-FITC NPs illustrating no macropinocytic uptake of NPs in this cell line.

FIG. 13 shows results of cell viability studies for exposure of SNU-5 cells to Co-Alb NPs.

### DETAILED DESCRIPTION OF PREFERRED EMBODIMENTS

The present disclosure now will be described more fully hereinafter with reference to the accompanying drawings, in which one, but not all embodiments of the inventions are illustrated. Indeed, these aspects may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The compounds may be referred to herein as “degradable” compounds or “labile” compounds, but neither term should be viewed as expressly limiting the scope of the compounds. Rather, the terms “degradable” and “labile” merely are used to describe the nature of the compounds, in that the inventive compounds are stable under one or more defined conditions but, under one or more different specified conditions, the compounds will undergo a chemical transformation (e.g. cleavage). This transformation may be exemplified by the breaking of one or more bonds within the compound that causes the compound to become fragmented. The transformation also may be exemplified by the partial or complete solubilization of the compound under the specified conditions. Accordingly, the terms “degradable” and “labile” may mean the compounds are subject to being transformed by a variety of means, and a skilled person viewing the present description would be able to envision a variety of methods whereby the inventive compounds could be degraded according to the various uses described herein, and all of such methods are encompassed by the present invention. In various embodiments, the degradation may be dependent upon one or more of the following conditions: pH; radiation; ionic strength; oxidation; reduction; temperature; an alternating magnetic field; an alternating electric field; combinations thereof; or the like.

In certain embodiments, the compounds of the present disclosure may be described as “reductively labile compounds”. A reductively labile compound is understood to mean a compound that may be chemically transformed (as described above) in relation to a change in reduction potential. Accordingly, a reductively labile compound may be stable at a potential below a certain value but degrade when the potential is raised above the certain value. Likewise, a reductively labile compound may be stable at a potential above a certain value but degrade when the potential is lowered below the certain value.

In one embodiment, the compounds may be described as “pH labile compounds” or “acid labile compounds.” A pH labile compound is understood to mean a compound that may be chemically transformed (as described above) in relation to a change in pH. Accordingly, a pH labile compound may be stable at a pH below a certain value but degrade when pH is raised above the certain value. Likewise, a pH labile compound may be stable at a pH above a certain value but degrade when pH is lowered below the certain value. In a specific embodiment, an acid labile compound is stable above a pH of 7.9, 7.8, 7.7, 7.6, 7.5, 7.4, 7.3, 7.2, 7.1, or 7.0 but degrades below the specified value. In other embodiments, an acid labile compound can comprise a compound that is stable at a pH above about 7.5, above about 7, or above about 6.5 but degrades below this value. In specific embodiments, a pH labile compound may be described as being degradable at cellular pH conditions. For example, in some embodiments, the (and compositions and particles incorporating the compounds) particularly may be designed to degrade under pH conditions typically found in cell endosomes.

According to certain embodiments, a degradable molecule according to the present disclosure may have a structure according to Formula (Ia),

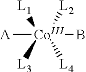

wherein:
A and B are the same or different and contain NH₃, NH₂R, NHR₁R₂, NR₁R₂R₃, OH₂, OHR, OHR₁R₂, SH₂, SHR₁, SHR₂, PH₃, PH₂R₁, PHR₁R₂, PR₁R₂R₃; R₁, R₂, and R₃ are the same or different and are optionally substituted straight or branched chain C₁-C₆ alkyl, alkenyl, or alkynyl; optionally substituted C₃-C₆ cycloalkyl, cycloalkenyl, cycloalkynyl, or hydrogen; optionally substituted phenyl or benzyl; or optionally substituted polyether or polyester; Co may contain up to four other bound ligands (L₁-L₄) including H, X (X being F, Cl, Br, or I), OR₂, NR₃, OR, SR, NR₂, PR₂, ER₃, EX₃ (E being Si, Ge, or Sn), CH₃ (or other alkyl group), η¹ to η⁷-aryl, η¹ to η⁷-alkenyl, η¹ to η²-alkynyl, η¹ to η²-acyl, η²-ketone, terminal carbene, terminal carbine, CO, CNR, N₂, NO, N₂R, NR, PR₃, PX₃, AsR₃, SbR₃, NR₃, RNCR₂, RCN, ether, thioether, N, O, or O₂.

The use of the phrase “optionally substituted” herein indicates that each C atom includes the appropriate number of H atoms to equal four bonds per carbon, or one or more H atoms may be replaced by a substituent. Preferred substituents are straight or branched chain C1-C4 alkyl, alkenyl, or alkynyl groups.

A polymerizable group, as used herein is understood to be any group that facilitates polymerization of the overall molecule to which it is attached, such as through reaction with another identical molecule (e.g., homopolymerization) or a different molecule (e.g., co-polymerization). In specific embodiments, a polymerizable group is a group that facilitates polymerization to form a homopolymer of repeating identical subunits. Thus, in certain embodiments, the compounds can be referred to as oligomers comprising polymerizable functional groups. Non-limiting examples of polymerizable groups useful according to the present invention include groups comprising a terminal C═C bond and groups comprising a C═O bond. In particular embodiments, polymerizable groups in the compounds comprise groups that are UV polymerizable (i.e., wherein polymerization proceeds upon application of ultraviolet light stimulus). Non-limiting examples of UV polymerizable groups that are useful include acrylate and methacrylate groups (e.g., groups comprising a moiety from acrylic acid or methacrylic acid). Yet further examples of polymerizable groups that are useful include maleimide, acrylamide, and methacrylamide groups. In some embodiments, the polymerizable group on the compound of Formula (1) is selected from the group consisting of an acrylate moiety, a methacrylate moiety, an epoxy moiety, an amino moiety, a carboxylic moiety, an anhydride moiety, a maleimide moiety, an isocyanate moiety, an olef[iota]nic moiety, a styrenic moiety, an acrylamide moiety, a methacrylamide moiety, and combinations thereof. It is understood that the foregoing list is only exemplary, and any polymerizable group for use as the A or B component are fully encompassed.

Co may contain up to four other bound ligands (L₁-L₄), as shown below:

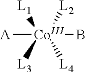

L₁-L₄ can be NH₃, NH₂R, NHR₁R₂, NR₁R₂R₃, OH₂, OHR, OHR₁R₂, SH₂, SHR₁, SHR₂, PH₃, PH₂R₁, PHR₁R₂, PR₁R₂R₃; R₁, R₂, and R₃ are the same or different and are optionally substituted straight or branched chain C1-C6 alkyl, alkenyl, or alkynyl; optionally substituted C3-C6 cycloalkyl, cycloalkenyl, cycloalkynyl, or hydrogen; optionally substituted phenyl or benzyl; or optionally substituted polyether or polyester; Co may contain up to four other bound ligands (L₁-L₄) including H, X (X being F, Cl, Br, or I), OR₂, NR₃, OR, SR, NR₂, PR₂, ER₃, EX₃ (E being Si, Ge, or Sn), CH₃ (or other alkyl group), η¹ to η⁷-aryl, η¹ to η⁷-alkenyl, η¹ to η²-alkynyl, η¹ to η²-acyl, η²-ketone, terminal carbene, terminal carbine, CO, CNR, N₂, NO, N₂R, NR, PR₃, PX₃, AsR₃, SbR₃, NR₃, RNCR₂, RCN, ether, thioether, N, O, or O₂.

The linkages between Co, A and B, as well as Co and L₁-L₄ can all be designed to respond to stimuli such as changes in pH and reduction potential, or external stimuli such as applied electromagnetic radiation. Therapeutic agents can be entrapped within the crosslinked molecules or covalently attached to the molecules though additional stimuli-responsive linkages. Also, the degradable molecules themselves can act as the therapeutic agents.

The present disclosure provides compositions comprising the degradable molecules of the invention. In certain embodiments, the compositions particularly may comprise a matrix material and a cargo component. The cargo component may be a drug, a therapeutic material, or any other compound capable of being physically or chemically combined with the matrix material. The matrix material may be at least partially formed using the degradable molecules of the invention, or the degradable molecules may be otherwise associated with the matrix material (e.g., attached to a surface of a particle formed from the matrix material). Thus, the composition provides for delivery and release of the cargo component through degradation of the molecules in the composition.

One embodiment pertains to a composition comprising a cargo component and a matrix material, the matrix material comprising degradable molecules having the structure of Formula (Ia), as described above. In specific embodiments, the composition may comprise about 0.1% to about 50% by weight of the matrix material, about 50% to about 99.9% by weight of the matrix material, or about 10% to about 90% by weight of the matrix material.

In some embodiments, the matrix material may be a co-polymer comprising the degradable molecules and one or more co-monomers. In further embodiments, the matrix material may comprise one or more polymers cross-linked with the degradable molecules. The composition also may comprise further components, such as a polymerization initiator (e.g., a photoinitiator). In other embodiments, the matrix material specifically may comprise a biodegradable polymer.

The cargo component may be associated with the matrix material via a variety of means. For example, the cargo component may be encapsulated by the matrix material, the cargo component may be the metal complex used for crosslinking, may be physically blended with the matrix material, and/or the cargo component may be covalently bonded to one or more functional groups present on the matrix material. In one embodiment, the matrix material may be in the form of a particle having the degradable molecules covalently bonded to one or more functional groups present on an exposed surface of the particle, and the cargo component may be attached to the matrix particle via the degradable molecules.

In certain embodiments, the composition may be provided in the form of discrete particles. Thus, the composition may be formed into discrete particles of micro- and nano-scale dimensions. Such particles may be administered directly to a site where it is desirable for the cargo component to be released. In some embodiments, the discrete particles may be incorporated into pharmaceutical formulations.

Nanoparticles are as solid colloidal particles ranging in size from about 20 nm to about 200 nm in diameter. They accumulate passively in solid tumors by enhanced permeation and retention (EPR) effect. Their sub-cellular dimensions allow for the delivery of specific materials (i.e. genes) directly to individual cells. Nanoparticles consist of an active agent (such as small molecule drug or biologic therapeutic such as protein-based drugs or nucleic acids, also called a “cargo”) either dissolved, entrapped, and/or encapsulated, or to which the active agent is adsorbed or attached. This encapsulation process enhances both the solubility and stability of the drugs and can also improve their pharmacokinetic properties. Targeted drug delivery is applicable to not only a wide variety of cancers, but also, auto-immune diseases such as rheumatoid arthritis, infectious diseases, AIDS, diabetes, cardiovascular diseases, and many others.

Accordingly, certain embodiments are directed to a particle comprising a degradable molecule having the structure of Formula (Ia), as described herein. The particle further may comprise a cargo component associated with the particle. Also, the particle may be a surface-activated particle. Specifically, the particle may have an exposed surface with a degradable molecule attached thereto and including a reactive group that makes the particle activated in that it is ready to receive a further component (e.g., a cargo component) that will covalently attach to the particle via the degradable compound.

A pharmaceutical formulation may comprise a pharmaceutically acceptable carrier, a pharmaceutical material, and a degradable molecule having the structure of Formula (Ia), as described herein. In particular, the formulation may comprise a matrix material including the degradable compound. Still further, the matrix material may be in the form of a particle. In such embodiments, the pharmaceutical material may be associated with the particle (e.g., attached to an exposed surface of the particle via the degradable compound and/or at least partially encapsulated by the particle). The pharmaceutical material may comprise one or more pharmaceutically active therapeutic agents or drugs. In additional embodiments, the pharmaceutical formulation may include non-pharmaceutically active components such as negatively charged components, negatively charged surfactants, negatively charged emulsifiers, positively charged components, excipients, adjuvants, stabilizers, diluents, carriers, lubricating agents, wetting agents, preserving agents, sweetening agents, flavoring agents, antioxidants, buffers, bacteriostats, solutes, aqueous suspensions, non-aqueous suspensions, solubilizers, thickening agents, sterile powders, tonicity modifiers, or combinations thereof.

In other embodiments, a composition may comprise a first particle type and a second particle type, each particle type comprising a matrix material and a cargo component, and the matrix material of at least one particle type comprising a degradable compound having the structure of Formula (Ia), as described herein.

In particular embodiments, the composition can vary by altering various components of the particles. For example, the first particle type can differ from the second particle type in one or more of the matrix material and the cargo component. Specifically, the polymeric makeup of the matrix material in each particle type may differ, and/or the cargo component used in each particle type may differ, and/or the degradable molecule used in each particle type may differ.

In another aspect, the present disclosure also provides methods of targeted drug delivery and methods of treatment of a subject. The wide applicability of the degradable particles described above makes them particularly useful in treating a wide variety of conditions and diseases. Virtually any drug or therapeutic agent may be formed into the inventive, degradable particles. Accordingly, certain embodiments pertain to methods of treating a patient comprising administering to the patient a composition according to the present disclosure (particularly in the form of discrete particles). Preferably, the composition comprises a drug or therapeutic agent known to prevent, treat, cure, or ameliorate a disease or condition. Thus, in one embodiment, the method may comprise administering to a patient a composition comprising a pharmaceutical material, and a degradable molecule having the structure of Formula (Ia), as described herein.

Aspects of the present disclosure present a novel strategy that can be used to crosslink protein to form nanoparticles ranging from 10 to 500 nm in size. The method preferably utilizes a labile Co²⁺ complex to crosslink lysine residues on adjacent proteins that can then be “locked” into conformation by oxidation to an exchange inert Co³⁺ complex. The coordination chemistry itself has ties dating back to the father of inorganic chemistry, Alfred Werner. The oxidized particles are stable for weeks in phosphate buffered saline (PBS) and cell culture media containing 10% fetal bovine serum, but degrade rapidly (<5 h) under reducing conditions. These particles are attractive as a drug delivery vector because the particles should remain intact in circulation, but degrade rapidly upon entry into a hypoxic environment, such as a tumor region, or cytosol, which is significantly more reducing in nature compared to extracellular space, allowing for the targeted release of encapsulated therapeutic.

### Example 1

### Synthesis of Albumin Nanoparticles

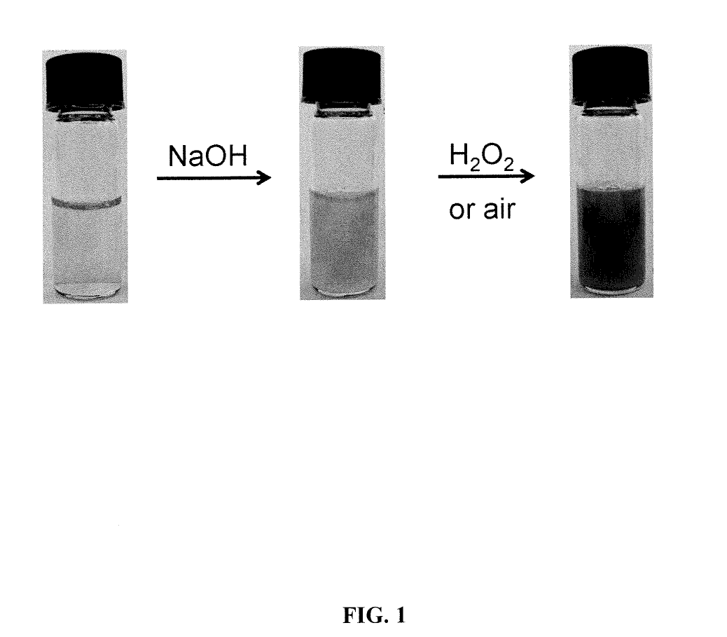

FIG. 1 shows the synthesis of albumin nanoparticles using cobalt(II) chloride, as follows: albumin+Cobalt chloride (left), co(II)-crosslinked albumin nanoparticles formed when solution pH is raised (center), and conversion of co(II)-crosslinked albumin nanoparticles to co(III)-crosslinked albumin nanoparticles (right).

More specifically, an aqueous solution (1.125 mL) containing bovine serum albumin (10 mg, Sigma-Aldrich, cat. #A2153-10G, lot #041M1816V) and cobalt(II) chloride hexahydrate (11.1 mM, Alfa Aesar, cat. #11344, lot #I18N25) was prepared in a glass vial (ChemGlass, cat. #CV-1256-1545), which was a faintly pink but clear solution (FIG. 1). While sonicating (Branson, model #2510), NaOH (50 μL, 0.25 M in water) was added at which time the solution became turbid and faintly blue (FIG. 1). The solution was allowed to stand undisturbed for 15 min. Dynamic light scattering (DLS, Microtrac Nanotrac Ultra) was used to measure particle size where the volume average particle size was 332 nm (standard deviation=31 nm). The nanoparticles were centrifuged at ˜21,000×g (Eppendorf, model #5810R) for 5 s and the supernatant removed. The particles were re-dispersed in 1.0 mL of DI water and centrifuged again at ˜21,000×g for 5 s. The supernatant was removed and the particles were re-dispersed in 1.0 mL of DI water. Hydrogen peroxide (20 μL of a 30% solution, Mallinckrodt AR, cat. #5240, lot #5240Y23470) was added while sonicating and the solution was thoroughly mixed by pipetting. The turbidity of the solution did not change, but the color changed from faint blue to a much more intense yellow-brown (FIG. 1). The solution was allowed to stand undisturbed for 30 min. The particles were centrifuged at ˜21,000×g for 5 s and the supernatant removed. The particles were re-dispersed in 1.0 mL of DI water and centrifuged again at ˜21,000×g for 5 s. The supernatant was removed and the particles were re-dispersed in 1.0 mL of DI water. DLS was used to measure particle size where a diameter of 262 nm (SD=39.5 nm) was obtained. Similar results were obtained upon scaling the reactants by a factor of 7×. Particles 20-500 nm in diameter were synthesized by changing the amounts of cobalt chloride and NaOH used in the reaction.

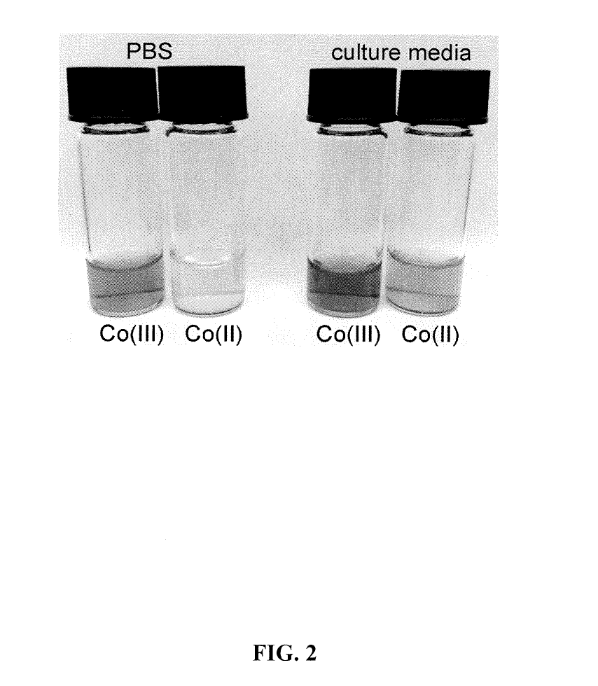

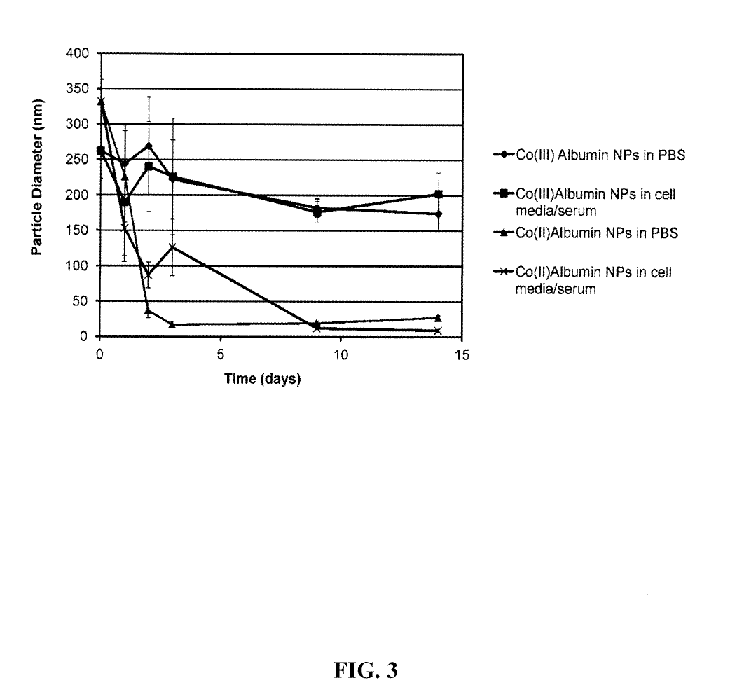

Nanoparticle Stability. Four samples containing 1 mL of the nanoparticle solutions obtained above both before (2 samples) and after (2 samples) addition of H₂O₂ were centrifuged at ˜21,000×g for 5 s and the supernatants removed. The resulting solids were re-dispersed in 1.0 mL of either 10 mM PBS (phosphate buffer saline) or Roswell Park Memorial Institute medium (RPMI)-1640 (ATCC, cat. #30-2001, lot #60488021, bottle #04041) containing 10% fetal bovine serum (ATCC, cat. #30-2020). The solutions were incubated at 37° C. with 5% CO₂ without agitation. FIG. 2 shows the solutions incubated for 14 days at 37° C. with no stirring under the indicated conditions. Yellow-brown color of co(iii)-crosslinked albumin nanoparticles (“NPs”) is clearly evident under both sets of reaction conditions. Particle size was measured periodically by DLS (FIG. 3). FIG. 3 shows the stability of the particle solutions over time. Particle size decreased significantly over 4 days for samples not oxidized with H₂O₂ (presumably containing Co(II)) whereas little change in size was observed over 14 days for solutions that had been oxidized with H₂O₂ (presumably containing Co(III)).

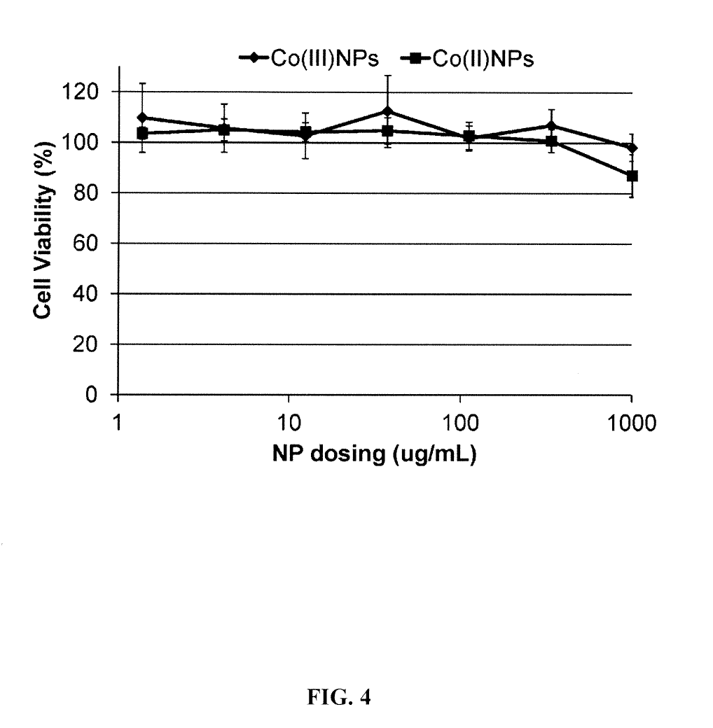

Cytotoxicity. SNU-5 cells were purchased from ATCC (cat. #CRL-5973), and maintained in Iscove's Modified Dulbecco's Medium (IMDM) (ATCC, cat. #30-2005) with 10% FBS (Fisherbrand Research Grade Fetal Bovine Serum, cat. #03-600-511). Cells (50,000/well) were seeded on 96-well plates and the desired particle amounts were added to the wells. The plates were incubated for an additional 24 h at 37° C. (5% CO₂). After incubation, cell viability was evaluated using MTT. MTT (Sigma-Aldrich, cat. #M2128-16, lot #MKBN7264B) dissolved in culture media (5 mg/mL) was added to each well (25 μL/well). The cells were incubated for 4 h at 37° C. (5% CO₂) after which time 0.08 M HCl in 2-propanol (100 μL/well) was added. Light absorption was measured on a Synergy 2 multi-mode microplate reader (BioTek, Synergy 5). The viability of the cells exposed to particles was expressed as a percentage of the viability of cells grown in the absence of particles on the same plate. FIG. 4 shows cytotoxicity of co-crosslinked albumin nanoparticles in SNU-5 cells. Very little toxicity was observed even at the highest particle dosing of 1 mg/mL (FIG. 4).

### Example 2

### Synthesis of Chitosan Nanoparticles

An aqueous solution (1.125 mL) containing chitosan oligosaccharide lactate (1 mg, Sigma-Aldrich, cat. #523682-1G, lot #MKBB2183V) and cobalt(II) chloride hexahydrate (11.1 mM, Alfa Aesar, cat. #11344, lot #118N25) was prepared in a glass vial (ChemGlass, cat. #CV-1256-1545), which was a faintly pink but clear solution. While sonicating (Branson, model #2510), NaOH (50 μL, 0.25 M in water) was added at which time the solution became turbid and faintly blue. The solution was allowed to stand undisturbed for 15 min. Dynamic light scattering (DLS, Microtrac Nanotrac Ultra) was used to measure particle size where the volume average particle size was 332 nm (standard deviation=31 nm). The nanoparticles were centrifuged at ˜21,000×g (Eppendorf, model #5810R) for 5 s and the supernatant removed. The particles were re-dispersed in 1.0 mL of DI water and centrifuged again at ˜21,000×g for 5 s. The supernatant was removed and the particles were re-dispersed in 1.0 mL of DI water. Hydrogen peroxide (20 μL of a 30% solution, Mallinckrodt AR, cat. #5240, lot #5240Y23470) was added while sonicating and the solution was thoroughly mixed by pipetting. The turbidity of the solution did not change, but the color changed from faint blue to a much more intense yellow-brown. The solution was allowed to stand undisturbed for 30 min. The particles were centrifuged at ˜21,000×g for 5 s and the supernatant removed. The particles were re-dispersed in 1.0 mL of DI water and centrifuged again at ˜21,000×g for 5 s. The supernatant was removed and the particles were re-dispersed in 1.0 mL of DI water. DLS was used to measure particle size where diameters ranging from 50-500 nm were synthesized by changing the amounts of cobalt chloride and NaOH used in the reaction.

### Example 3

### Bioconjugation of Peg Chains to Transferrin

A borate buffered solution (47.6 mM, 1.05 mL) containing cobalt(II) chloride hexahydrate (0.95 mM, Alfa Aesar, cat. #11344, lot #18N25) and H₂N-PEG-COOH HCl (5 mg, Rapp Polymere, cat. #1350002032, no. 128.710) was prepared in a glass vial (ChemGlass, cat. #CV-1256-1545). While sonicating (Branson, model #2510), human transferrin (0.5 mL of a 1 mg/mL aqueous solution, Sigma-Aldrich, cat. #T0665-50MG, lot #SLBC 1149V) was added. The solution had no discernible color and was allowed to stand undisturbed for 15 min. Dynamic light scattering (DLS, Microtrac Nanotrac Ultra) was used to measure particle size where the volume average particle size was 13.6 nm (standard deviation=4.2 nm). Hydrogen peroxide (20 μL of a 30% solution, Mallinckrodt AR, cat. #5240, lot #5240Y23470) was added while sonicating and the solution was thoroughly mixed by pipetting. The solution became faintly yellow but with no turbity. The solution was allowed to stand undisturbed for 30 min.

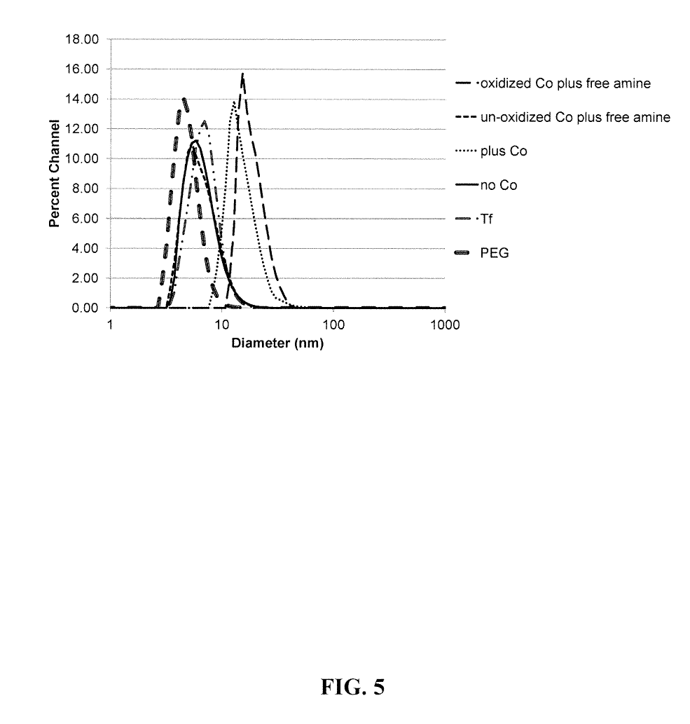

Analysis of PEGylation reaction by DLS. The average diameter of both human transferrin (6.8 nm, SD=1.8 nm) and H₂N-PEG-COOH (4.8 nm, SD=1.2 nm) were measured by DLS (FIG. 5). FIG. 5 shows particle size analysis of the pegylation (PEG, poly(ethylene glycol)) of transferrin (T_{f}). Mixtures of human transferrin and H₂N-PEG-COOH displayed a monomodal distribution with an average diameter of 6.5 nm (SD=1.9 nm). Addition of transferrin to a solution containing cobalt and H₂N-PEG-COOH led to an increase in particle size to 14.8 nm (SD=4.0 nm) indicating successful conjugation of the two molecules. Addition of ethanolamine (50 mM) at this point led to a decrease in particle size back to 6.4 nm (SD=2.0) thereby degrading the prodrug linkage due to facile exchange of the Co amino ligands. If the sample was first oxidized for 30 min with H₂O₂ (converting Co(II) to Co(III)) before the addition of ethanolamine, the average diameter of the transferrin-PEG conjugate increased slightly to 18.3 nm (SD=4.5 nm) and the prodrug linkage remained intact. These results were in accord with the expected lability of the Co-complexes in both oxidation states.

### Example 4

### Albumin Nanoparticles Loaded with SN-38

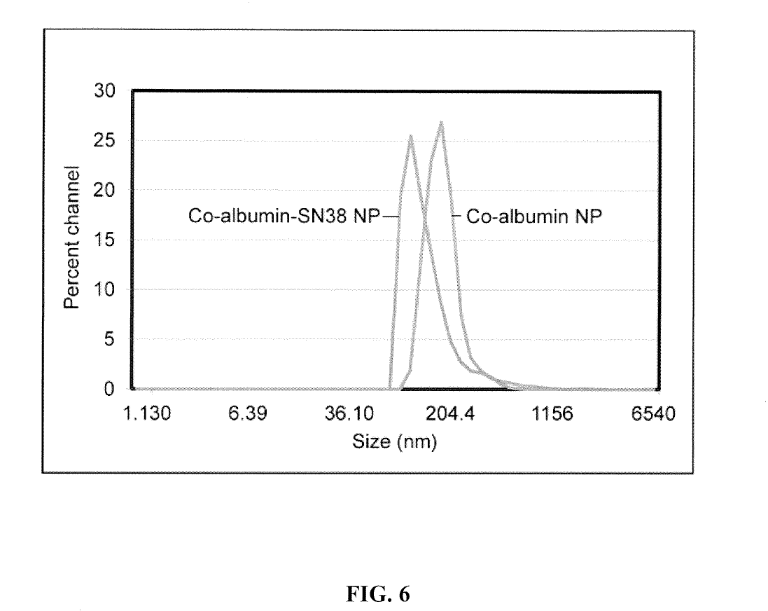

Bovine serum albumin was prepared in ultrapure water as a 10 mg/mL solution. Cobalt(II) chloride hexahydrate (100 mM) and 25 mM of NaOH solution was also prepared in ultrapure water. In a small glass vial, 200 uL of bovine serum albumin (10 mg/mL aqueous) was mixed with 50 uL of NaOH (25 mM) and the solution was colorless. Cobalt(II) chloride hexahydrate (50 L, 100 mM) was added and the solution immediately turned blue and became turbid. The solution was sonicated for 5 s and left undisturbed for 15 minutes at room temperature. Hydrogen peroxide (5 L, 30% aqueous) was added to the solution followed by 1 mL of ultrapure water. The solution immediately turned yellow without any changes in turbidity. The solution was centrifuged (Eppendorf, model #5810R) for 1 minute at 21,000×g and the supernatant was discarded. The pellet was then resuspended into 1 mL of water, centrifuged at 21,000×g and the supernatant was discarded. The pellet was suspended in water and dynamic light scattering (DLS, Microtrac Nanotrac Ultra) was used to measure the size of the nanoparticle. The nanoparticles were 140-200 nm in diameter (FIG. 6).

SN-38 (7-ethyl-10hydroxycampothecin, TCI Development Co., Shanghai, China) was dissolved in dichloromethane/ethanol (1:1) at 10 mg/mL. An emulsification/solvent evaporation method was used to incorporate SN-38 into Co-Alb NPs, which was accomplished by mixing 5 mL of a Co-alb NP solution with 2.5 mL of the SN-38 solution. Sonication was applied for 2-3 minutes followed by stirring at RT for three hours to allow all the organic phase to evaporate. The solution was then centrifuged at 21,000×g for 5 minutes. The supernatant, which contained unbound SN-38 was removed and analyzed to determine drug loading. The pellet was then washed three times with ultrapure water. The particles were re-suspended in water and analyzed by DLS (FIG. 6). FIG. 6 shows the Dynamic Light Scattering results for Co-Alb SN-38 NPs.

To determine SN-38 loading, the supernatants collected after centrifugation were analyzed by UV/Vis (Synergy 2, Biotek USA) based on a calibration curve of SN-38 (380 nm). Drug loading capacity and encapsulation efficiency was calculated according to Equations 1 and 2 below.

$$Encapsulation Efficiency = \frac{Total drug - residual drug}{Total drug} \times 100$$

$$Drug loading capacity = \frac{Loaded drug}{Total weight of NPs} \times 100$$

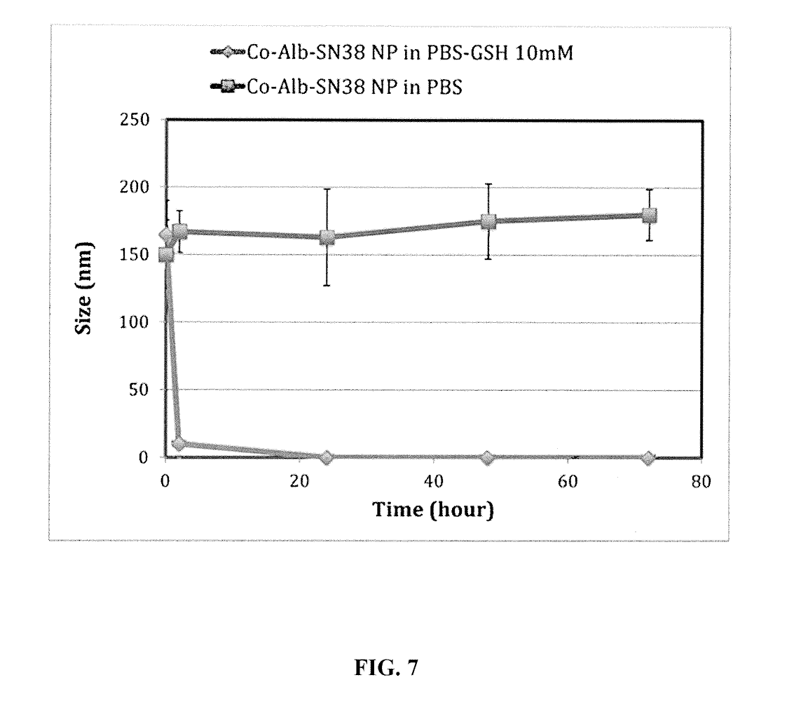

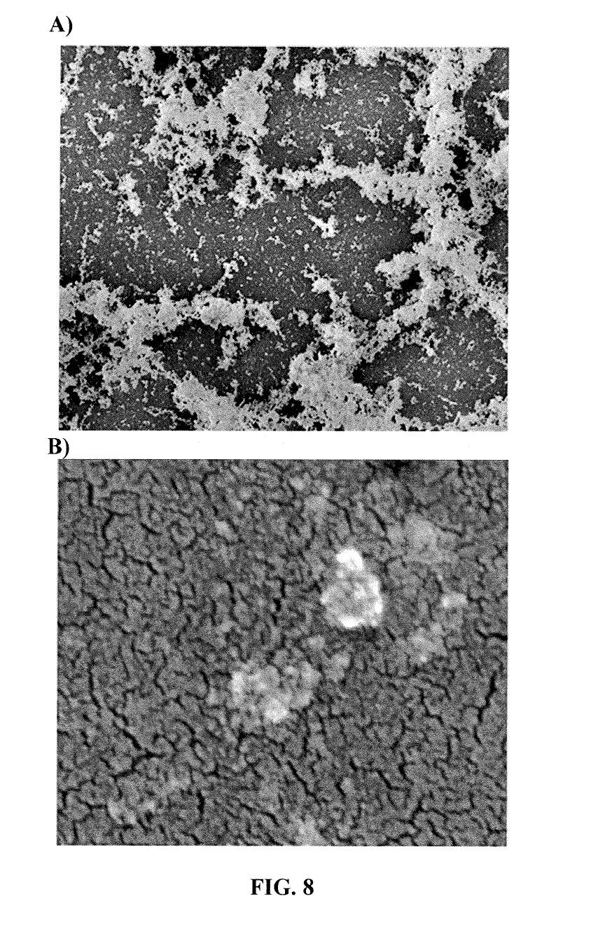

Encapsulation efficiency was observed to be at 94% and loading capacity was approximately 31%. SN-38 loaded Co-Alb NPs exhibited minimal changes in size upon incubating in PBS; however when incubated in PBS plus reduced glutathione (GSH 10 mM), the nanoparticles degraded rapidly (FIG. 7). FIG. 7 shows the stability of SN-38 loaded Co-Alb NPs. Representative SEM images of the Co-Alb NPs are shown in FIG. 8.

### Example 5

### In Vitro Evaluation of Uptake and Toxicity

This example presents the in vitro characterization of particle uptake for fluorescently labeled cobalt crosslinked albumin nanoparticles (Co-Alb-FITC NPs) as well as preliminary toxicity data. Generally, Co-Alb-FITC NPs were incubated with gastric carcinoma cells (SNU-5) where uptake proceeded rapidly as measured by image-based flow cytometry. Upon pre-incubation of the cells with a known inhibitor of macropinocytosis, 5-(N-ethyl-N-isopropyl)amiloride (EIPA), a dramatic reduction in particle uptake was observed indicating that macropinocytosis was the mechanism responsible for particle internalization. Uptake by cells incubated with Co-Alb-FITC NPs only was >8 times higher than uptake of the same concentration Co-Alb-FITC NPs in the presence of 100 μM EIPA. Incubation of SNU-5 cells with free FITC-Alb displayed similar inhibitor-dependent behavior. Analogous experiments in Jurkat T cell lymphocytes resulted in low levels of particle uptake that were unaffected by inhibitor. High levels of non-specific uptake of other targeted particle formulations were also observed in SNU-5 cells suggesting that this particular cell line possesses high marcopinocytic activity. Cancer cells harboring mutated Ras (>20% of all cancers) were recently reported to take up serum proteins via macropinocytosis to satisfy metabolic demands. Since SNU-5 cells are not known to harbor a Ras mutation, the findings reported here may indicate that other cancer cell types rely on macropinocytosis for nutrient uptake, which would have important implications for “active” nanoparticle targeting strategies. Co-Alb-FITC NPs were found to be highly biocompatible with no toxicity observed at high particle dosing (1 mg/mL).

By way of background, the rapid proliferation of cancers cells requires a significant amount of nutrients the sources of which have been the subject of a large body of research. More effective treatments could be realized if the sources of nutrients could be identified and then systematically cut off from the tumor. Recent studies have now clearly demonstrated that a host of cancer cell types utilize serum proteins as a major source of the required nutrients and building blocks, which was found to be associated with a Ras mutation present in ˜20% of all cancers. Such cells are capable of engulfing large amounts of protein via macropinocytosis, which is a non-specific process that lacks the requirement for engaging a particular receptor on the cell surface and can form vesicles up to 5 μm in diameter. These findings will likely have important implications in the field of nanoparticle therapeutics given the efforts currently devoted to elucidating cellular targeting/internalization strategies and the ongoing debate over their effectiveness. Particles are already known to accumulate at tumor sites due to the enhanced permeability and retention (EPR) effect, so an important question arises as to whether a targeting/internalization strategy will be necessary at all for tumors exhibiting this behavior. For example, while previous studies found no increase in accumulation of particles at tumor sites based on the presence or absence of targeting ligands, an increase in particle internalization was observed for their transferrin receptor targeted particles. It should be noted however that the neuroblastoma cells like the ones used in the tumor models do not typically possess mutated Ras, so it is unclear whether differential uptake would have been observed in the presence of such a mutation.

General Materials and Methods.

Albumin-fluorescein isothiocyanate conjugate (cat. #A9771), bovine serum albumin (cat. #A2153), o-(2-aminoethyl)polyethylene glycol (MW 5K, cat. #672130), and thiazolyl blue tetrazolium bromide, MTT (cat. #M5655) were from Sigma-Aldrich, fetal bovine serum (cat. #03-600-511), 5-(N-ethyl-N-isopropyl)amiloride, EIPA (cat. #37-781-0) were from Fisher Scientific, SNU-5 cells (cat. #ATCC® CRL-1420) Jurkat cells (cat. #ATCC® TIB-152), RPMI-1640 medium (cat. #ATCC® 30-2001), IMDM medium (cat. #ATCC® 30-2005) were from ATCC, Met (L6E7) Mouse mAb (cat. #8741), and anti-mouse IgG (H+L), F(ab′)2 Fragment (Alexa Fluor® 488 Conjugate) (cat. #4408) were from Cell Signaling Technology, and Micromer®-redF (cat. #30-02-501) was from Micromod. All reagents were used without further purification. Flow cytometry was performed using either an Amnis FlowSight (EMD Millipore; Seattle, Wash.) or Guava EasyCyte 6.2L (EMD Millipore; Seattle, Wash.) instrument. For the FlowSight, spectral compensation was completed after analysis using an automated wizard and single color control samples in the IDEAS software. Prior to collecting samples, the performance of the FlowSight was validated using FlowSight calibration beads (EMD Millipore).

In order to examine particle uptake by image-based flow cytometry, fluorescently labeled particles were prepared by adding a commercially available fluorescein isothiocyanate (FITC)-albumin conjugate during nanoparticle synthesis to generate FITC-labeled Co-Alb NPs (Co-Alb-FITC NPs).

Synthesis of Co-Alb-FITC NPs.

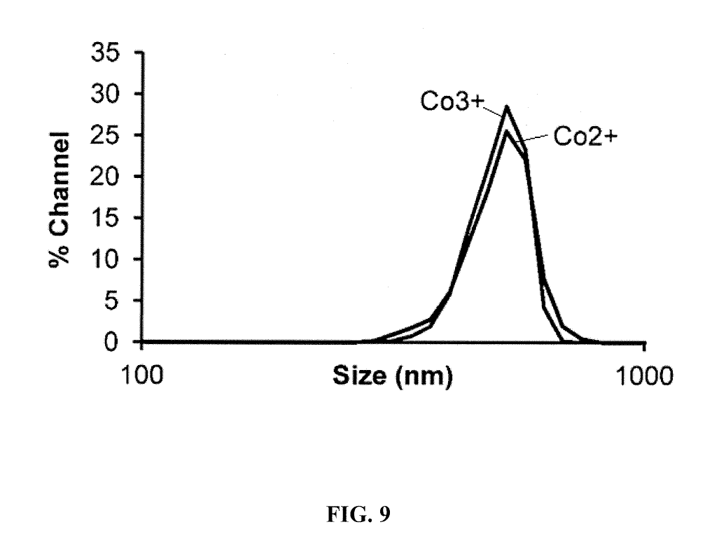

All solutions used in the synthesis of nanoparticles were freshly prepared and used within one week. A 1 mL aqueous solution containing 7.5 mg of bovine serum albumin-FITC conjugate and 2.5 mg of was prepared followed by the sequential addition of CoCl₂H₂O₆ (200 μL, 0.1 M in H₂O) and NaOH (50 μL, 0.25 M in H₂O) while sonicating. The solution was allowed to stand undisturbed for 10 min at room temperature and then centrifuged at 21,000×g for 30 s. The supernatant was removed and the particles were washed once with 1 mL ultra-pure water. After washing, the particles were re-dispersed in 1 mL of ultra-pure water and analyzed by dynamic light scattering (DLS). DLS results indicated a monodisperse population of particles with a number average diameter of 498 nm (FIG. 9). Hydrogen peroxide (0.2 μL, 30% v/v in H₂O) was added and the solution allowed to stand undisturbed for 10 min. The particles were then centrifuged at 21,000×g for 1 min, the pellet washed once with ultra-pure water, and re-dispersed in 1 mL of ultra-pure water. An average diameter of 493 nm was observed by DLS (FIG. 9), indicating no change in particle size upon oxidation of Co²⁺ to Co³⁺. Co-Alb-FITC NP solutions were used immediately or stored for up to 1 week in the dark at 4° C.

The particles utilized in these studies had an average diameter of ˜500 nm as determined by dynamic light scattering (FIG. 9) although other sizes as small as 10 nm can be synthesized as previously reported. An Amnis FlowSight image-based flow cytometer equipped with a 488 nm (60 mW) and Side Scatter (7.85 mW) (EMD Millipore; Seattle, Wash.) was used to analyze cell samples. The FlowSight uses a CCD camera system to simultaneously collect both quantitative fluorescence and image data. For uptake studies, SNU-5 cells were incubated with Co-Alb-FITC NPs in the presence or absence of a commonly used inhibitor of macropinocytosis, 5-(N-ethyl-N-isopropyl)amiloride (EIPA), followed by fixation, and analysis via image-based flow cytometry.

Uptake Experiments.

SNU-5 cells were cultured at 37° C. and 5% CO₂ in IMDM medium supplemented with 20% fetal bovine serum at cell densities between 1×10⁵ and 1×10⁶ cells/mL, and Jurkat cells were cultured at 37° C. and 5% CO₂ in RPMI-1640 medium supplemented with 10% fetal bovine serum at cell densities between 1×10⁵ and 1×10⁶ cells/mL. For nanoparticle uptake experiments, cells were transferred to serum-starved IMDM or RPMI-1640 medium (0.1% FBS) and incubated at 37° C. and 5% CO₂ for 10-16 h prior to dosing with nanoparticles. Cells (2 mL, 500,000 cells/mL) were incubated with the desired nanoparticle or control particle solution (100 μg/mL) with or without added EIPA for the times indicated. Cells were then washed twice with PBS, fixed for 12 min in 3.7% formaldehyde in PBS, washed twice with PBS, stained with NucBlue® Fixed Cell ReadyProbes® Reagent, and then washed twice more with PBS. Samples were either analyzed immediately or stored at 4° C. in the dark until analysis.

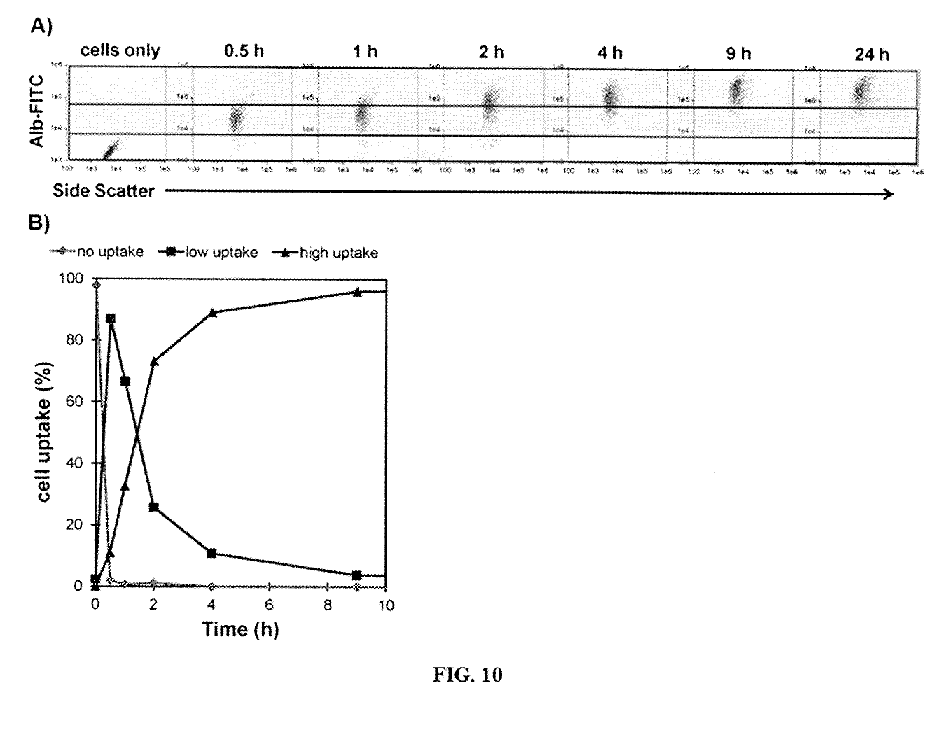

Rapid particle uptake was observed in the absence of EIPA with virtually all cells exhibiting an increase in FITC emission after just 30 min of exposure to Co-Alb-FITC NPs (FIG. 10(a)). FIG. 10(a) shows dot plots of side scatter vs. albumin-FITC fluorescence illustrating the kinetics of uptake of Co-Alb-FITC NPs (100 μg/mL dosing) by SNU-5 cells (cells exhibiting no (bottom), low (middle), or high (top) uptake). Representative FlowSight images of SNU-5 cells from the dot plots shown in FIG. 10(a) visually confirmed these results. Uptake was observed to increase steadily during the first ˜9 h at which point it appeared to reach saturation and no further uptake was observed upon prolonging exposure to particles (FIG. 10(b)). FIG. 10(b) shows the percentages of cells displaying no, low, or high uptake of Co-Alb-FITC NPs from FIG. 10(a). Table 1 below shows a complete listing of uptake percentages.

TABLE 1

| | | Form of | Incubation | | No | Low | High |
|---|---|---|---|---|---|---|---|
| | Cell | FITC- | time | EIPA | uptake | uptake | uptake |
| Sample | line | albumin | (h) | (μM) | (green) | (blue) | (red) |
| 1 | SNU-5 | CoAlb-FITC NPs | cells only | 0 | 97.8 | 2.2 | 0.0 |
| 2 | SNU-5 | CoAlb-FITC NPs | 0.5 | 0 | 2.0 | 87.0 | 11.0 |
| 3 | SNU-5 | CoAlb-FITC NPs | 1 | 0 | 0.6 | 66.7 | 32.6 |
| 4 | SNU-5 | CoAlb-FITC NPs | 2 | 0 | 1.1 | 25.7 | 73.1 |
| 5 | SNU-5 | CoAlb-FITC NPs | 2 | 25 | 1.6 | 54.9 | 43.5 |
| 6 | SNU-5 | CoAlb-FITC NPs | 2 | 75 | 2.1 | 81.6 | 16.2 |
| 7 | SNU-5 | CoAlb-FITC NPs | 4 | 0 | 0.0 | 10.9 | 89.1 |
| 8 | SNU-5 | CoAlb-FITC NPs | 9 | 0 | 0.1 | 3.9 | 96.0 |
| 9 | SNU-5 | CoAlb-FITC NPs | 24 | 0 | 0.0 | 2.7 | 97.2 |
| 10 | SNU-5 | FITC-Alb only | cells only | 0 | 97.2 | 2.8 | 0 |
| 11 | SNU-5 | FITC-Alb only | 8 | 0 | 40.6 | 45.4 | 13.9 |
| 12 | SNU-5 | FITC-Alb only | 8 | 75 | 71.3 | 11.8 | 16.9 |
| 13 | Jurkat | CoAlb-FITC NPs | cells only | 0 | 98.2 | 1.8 | 0 |
| 14 | Jurkat | CoAlb-FITC NPs | 9 | 0 | 4.5 | 94 | 1.5 |
| 15 | Jurkat | CoAlb-FITC NPs | 9 | 25 | 4.1 | 92.3 | 3.6 |

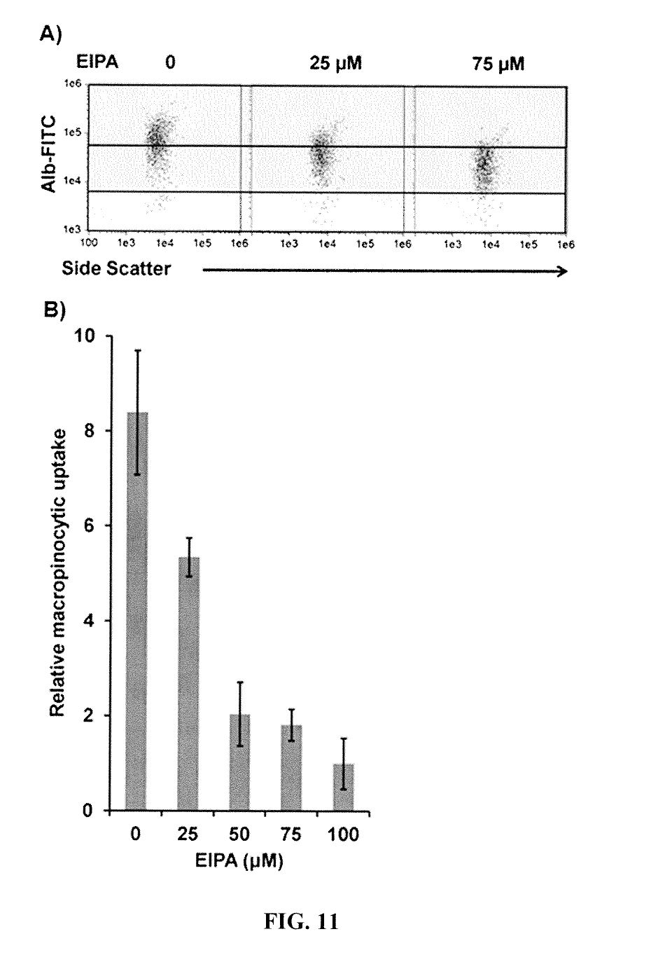

Cells pre-treated with EIPA for 5 min prior to Co-Alb-FITC NPs dosing displayed significantly reduced uptake (FIG. 11(a)) supporting the assertion that uptake was facilitated by macropinocytosis. FIG. 11(a) shows dot plots of side scatter vs. albumin-FITC fluorescence for SNU-5 cells incubated with various concentrations of EIPA illustrating the inhibitory effect on uptake of Co-Alb-FITC NPs after 2 h incubation (cells exhibiting no (bottom), low (center), or high (top) uptake). Representative FlowSight images of SNU-5 cells visually confirmed the information in FIG. 11(a) and Table 1 above includes the complete listing of percentages. The relative proportion of cells exhibiting high uptake was reduced from 73% in the absence of EIPA to 16% in the presence of 75 μM EIPA. EIPA was also found to reduce uptake in a dose-dependent manner with cells incubated in the absence of inhibitor exhibiting >8 times more macropinocytic uptake relative to cells pre-incubated with 100 μM EIPA (FIG. 11(b)). FIG. 11(b) shows relative macropinocytic uptake of Co-Alb-FITC NPs in the presence of varying amounts of an EIPA (4 h incubation time). Uptake values reported are relative to uptake at 100 μM EIPA. It should be noted that a slight decrease in cell viability was observed at 100 μM EIPA. The inhibitory effect of EIPA remained relatively constant over 8 h, but became greatly diminished at 24 h.

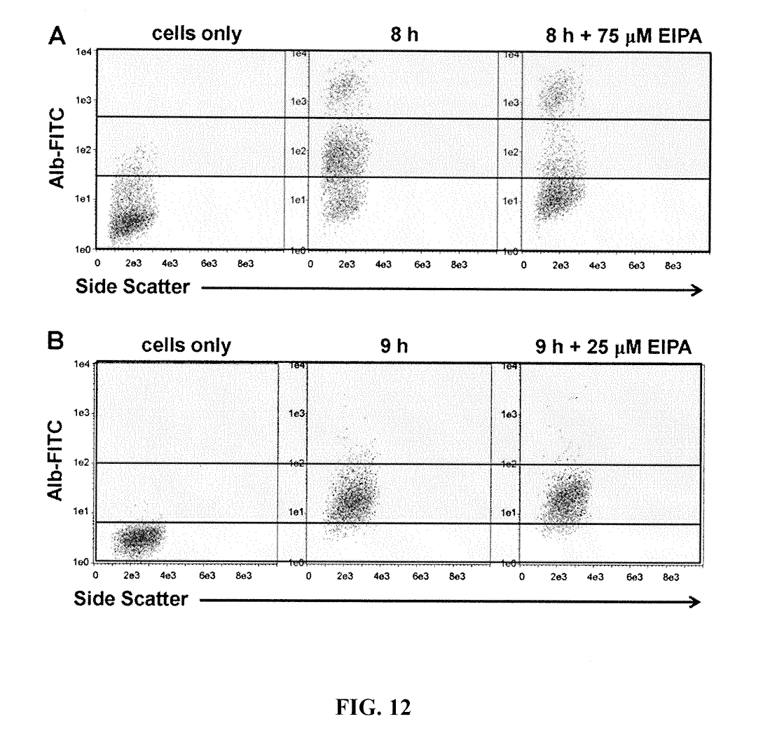

FIG. 12(a) shows dot plots of side scatter vs. albumin-FITC fluorescence for SNU-5 cells incubated with free FITC-albumin (100 μg/mL, no NPs) illustrating macropinocytic uptake of the free protein itself, and FIG. 12(b) shows dot plots of side scatter vs. albumin-FITC fluorescence for Jurkat cells incubated with Co-Alb-FITC NPs illustrating no macropinocytic uptake of NPs in this cell line. The dot plots show cells exhibiting no (bottom), low (center), or high (top) uptake. A complete listing of uptake percentages can be found in Table 1 above. Staining patterns similar to those for Co-Alb-FITC NPs were observed for SNU-5 cells incubated with free Alb-FITC, albeit less intense, that also showed a reduction in uptake in the presence of EIPA (FIG. 12(a)). These collective results clearly demonstrated a high degree of macropinocytic activity by SNU-5 cells. Another cell line utilized in our laboratory, Jurkat T lymphocyte cells, was also examined for particle uptake. Jurkat cells displayed very little binding/uptake of particles even at prolonged exposure with less than 2% exhibiting high uptake, and the small amount observed was largely un-affected by the presence of inhibitor (FIG. 12(b)) indicating that Jurkat cells displayed virtually no macropinocytic uptake.

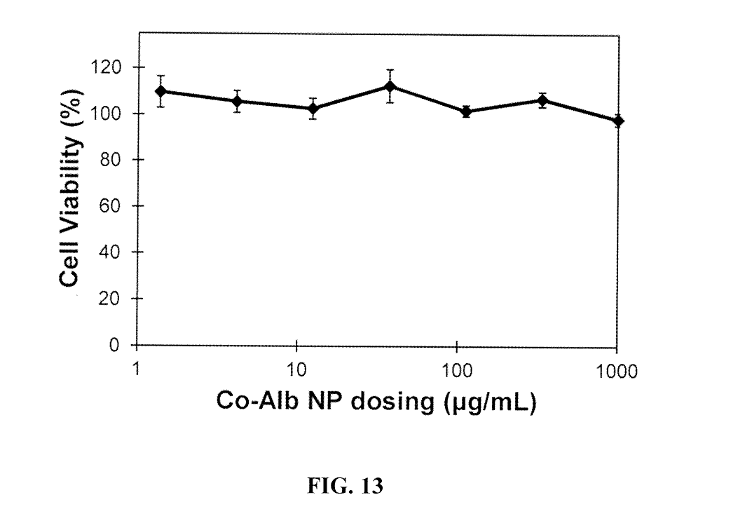

The inherent toxicity of Co-Alb NPs was measured in SNU-5 cells via a standard assay based on MTT. Cells were incubated with Co-Alb NPs for 48 h prior to conducting the MTT viability assay. FIG. 13 shows cell viability studies for exposure of SNU-5 cells to Co-Alb NPs demonstrating no toxicity from cobalt contained in the nanoparticles up to 1 mg/mL dosing. No significant toxicity was observed up to the highest dosing of nanoparticles (FIG. 13), which indicates a high degree of biocompatibility of the particles and no significant toxicity resulting from Co. With the biocompatibility of Co-Alb NPs now established, current efforts are focused on encapsulating a therapeutic either through physical encapsulation in the particles or more ideally through binding of an amine-containing drug to the Co complexes used as crosslinkers. For the latter, an amine-containing drug could be first reacted with Co²⁺ to form a prodrug, which could then be used to crosslink albumin into nanoparticles followed by oxidation to Co³⁺. This method would have the added advantage of a triggered release mechanism based on reduction of Co³⁺ to Co²⁺ to facilitate release rather than relying on the passive diffusion of a physically encapsulated drug from the particle.

Several important conclusions can be drawn from the results reported here. First, Co-Alb NPs are efficiently internalized by cancer cells that display high levels of macropinocytic uptake. Second, no toxicity was observed for Co-Alb NPs thereby establishing their biocompatibility and foreshadowing their potential as a drug delivery vector. Third, because SNU-5 cells are not known to harbor Ras mutations, the results could indicate that some non-Ras mutated tumor types also rely on macropinocytosis as a mechanism of cell survival. Tumors derived from such cells could conceivably take up large amounts of nanoparticles from circulation even in the absence of surface-bound targeting ligands, which would be significant benefit in the field of nanoparticle therapeutics.

### REFERENCES CITED

The following documents and publications are hereby incorporated by reference:

### U.S. PATENT DOCUMENTS

U.S. Patent Application Publication No. 2011/0123446

### OTHER PUBLICATIONS

Harris A N, et al., Beyond platinum: synthesis, characterization, and in vitro toxicity of Cu(II)-releasing polymer nanoparticles for potential use as a drug delivery vector. Nanoscale Res. Lett., 2011, Jul. 11, 6:445.
Lee E S, et al., Doxorubicin loaded pH-sensitive polymeric micelles for reversal of resistant MCF-7 tumor. J Control. Release. 2005, 103:405-418.
Lee E S et al., Polymeric micelle for tumor pH and folate-mediated targeting. J Control. Release. 2003, 91:103-113.
Maeda H, Wu J, Sawa T, Matsumura Y, Hori K. Tumour vascular permeability and the EPR effect in macromolecular therapeutics: a review. J. Control. Release 2000, 65:271-84.
Muthu M S and Singh S. Targeted nanomedicines: Effective treatment modalities for cancer, AIDS, and brain disorders. Nanomedicine. 2009, 4(1): 105-118.
Na K, et al., pH-sensitivity and pH-dependent interior structural change of self-assembled hydrogel nanoparticles of pullulan acetate/oligo-sulfonamide conjugate. J. Control. Release. 2004, 97:513-525.
Oh, K. T.; Yin, H. Q.; Lee, E. S.; Bae, Y. H., Polymeric nanovehicles for anticancer drugs with triggering release mechanisms. J Mater Chem 2007, 17, (38), 3987-4001.
Petros R A, DeSimone J M: Strategies in the design of nanoparticles for therapeutic applications. Nature Reviews Drug Discovery 2010, 9:615-627.
You, J-O et al., Bioresponsive matrices in drug delivery. J. Biol. Eng. 2010, 4:15.

What is claimed is:

1. A degradable molecule for targeted delivery of therapeutic agents comprising Formula Ia: 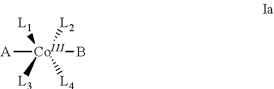
wherein:
A and B are the same or different and contain NH₃, NH₂R, NHR₁R₂, NR₁R₂R₃, OH₂, OHR, OHR₁R₂, SH₂, SHR₁, SHR₂, PH₃, PH₂R₁, PHR₁R₂, PR₁R₂R₃; R₁, R₂, and R₃ are the same or different and are optionally substituted straight or branched chain C1-C6 alkyl, alkenyl, or alkynyl; optionally substituted C3-C6 cycloalkyl, cycloalkenyl, cycloalkynyl, or hydrogen; optionally substituted phenyl or benzyl; or optionally substituted polyether or polyester; Co may contain up to four other bound ligands (L₁-L₄) including H, X (X being F, Cl, Br, or I), OR₂, NR₃, OR, SR, NR₂, PR₂, ER₃, EX₃ (E being Si, Ge, or Sn), CH₃ (or other alkyl group), η¹ to η⁷-aryl, η¹ to η⁷-alkenyl, η¹ to η²-alkynyl, η¹ to η²-acyl, η²-ketone, terminal carbene, terminal carbine, CO, CNR, N₂, NO, N₂R, NR, PR₃, PX₃, AsR₃, SbR₃, NR₃, RNCR₂, RCN, ether, thioether, N, O, or O₂.

2. The degradable molecule of claim 1, wherein one or more bonds to Co are labile in response to stimuli.

3. The degradable molecule of claim 2, wherein the one or more bonds to Co are labile in response to change in temperature, change in pH, change in reduction potential, applied electromagnetic radiation, or a combination thereof.

4. The degradable molecule of claim 1, further comprising one or more therapeutic agents attached at any available position through bonds that are labile in response to stimuli.

5. The degradable molecule of claim 4, wherein the bonds are labile in response to change in temperature, change in pH, change in reduction potential, or applied electromagnetic radiation.

6. A drug delivery composition comprising:
one or more degradable molecules of claim 1;
a matrix material; and
a cargo component.

7. The drug delivery composition of claim 6, wherein the matrix material is at least partially formed using the one or more degradable molecules.

8. The drug delivery composition of claim 6, wherein the one or more degradable molecules are attached to the matrix material.

9. The drug delivery composition of claim 6, wherein the matrix material comprises particles formed from the matrix material and wherein the one or more degradable molecules are attached to a surface of a particle formed from the matrix material.

10. The drug delivery composition of claim 6, wherein the composition comprises about 0.1% to about 50% by weight of the matrix material, about 50% to about 99.9% by weight of the matrix material, or about 10% to about 90% by weight of the matrix material.

11. The drug delivery composition of claim 6, wherein the matrix material comprises a co-polymer comprising the degradable molecules and one or more co-monomers.

12. The drug delivery composition of claim 6, wherein the matrix material comprises one or more polymers cross-linked with the degradable molecules.

13. The drug delivery composition of claim 6, further comprising a polymerization initiator.

14. The drug delivery composition of claim 6, wherein the matrix material comprises a biodegradable polymer.

15. The drug delivery composition of claim 6, wherein the cargo component comprises one or more therapeutic agents.

16. The drug delivery composition of claim 6, wherein the cargo component is encapsulated by the matrix material, is physically blended with the matrix material, or is covalently bonded to one or more functional groups present on the matrix material.

17. The drug delivery composition of claim 6, wherein the matrix material is in the form of a particle having the degradable molecules covalently bonded to one or more functional groups present on an exposed surface of the particle, and the cargo component is attached to the particle via the degradable molecules.

18. The drug delivery composition of claim 6, wherein the composition is in the form of discrete particles.

19. The drug delivery composition of claim 18, wherein the discrete particles comprise a first particle type and a second particle type, wherein each particle type comprises a matrix material and a cargo component, and wherein the matrix material of at least one particle type comprises the degradable molecules.

20. The drug delivery composition of claim 6, further comprising cell targeting components.

21. The drug delivery composition of claim 20 wherein the cell targeting components comprise nucleic acids, polypeptides, glycoproteins, carbohydrates, lipids, or combinations thereof.

22. The drug delivery composition of claim 20, wherein the cell targeting components comprise nucleic acid targeting moieties, protein targeting moieties, antibodies, carbohydrate targeting moieties, lipid targeting moieties, or combinations thereof.

23. A method for targeted drug delivery comprising administering the drug delivery composition of claim 6 to a subject.

24. A method for treatment of a subject comprising administering the drug delivery composition of claim 6 to the subject.

25. A pharmaceutical formulation comprising:
a pharmaceutically acceptable carrier;
a pharmaceutical material; and
one or more degradable molecules of claim 1.

26. The pharmaceutical formulation of claim 25, further comprising a matrix material, wherein the one or more degradable molecules are contained within the matrix material.

27. The pharmaceutical formulation of claim 26, wherein the matrix material is at least partially formed using the one or more degradable molecules.

28. The pharmaceutical formulation of claim 26, wherein the matrix material is in the form of a particle.

29. The pharmaceutical formulation of claim 25, wherein the pharmaceutical material is attached to an exposed surface of the particle via the degradable molecules or at least partially encapsulated by the particle.

30. The pharmaceutical formulation of claim 25, wherein the pharmaceutical material comprises one or more pharmaceutically active therapeutic agents.

31. The pharmaceutical formulation of claim 25, further comprising non-pharmaceutically active components.

32. The pharmaceutical formulation of claim 31, wherein the non-pharmaceutically active components comprise negatively charged components, negatively charged surfactants, negatively charged emulsifiers, positively charged components, excipients, adjuvants, stabilizers, diluents, carriers, lubricating agents, wetting agents, preserving agents, sweetening agents, flavoring agents, antioxidants, buffers, bacteriostats, solutes, aqueous suspensions, non-aqueous suspensions, solubilizers, thickening agents, sterile powders, tonicity modifiers, or combinations thereof.

33. A method for targeted drug delivery comprising administering the pharmaceutical formulation of claim 25 to a subject.

34. A method for treating a subject comprising administering the pharmaceutical formulation of claim 25 to the subject.

35. A drug delivery composition comprising:
one or more degradable molecules of claim 1; and
a matrix material.

36. The drug delivery composition of claim 35, wherein the matrix material is at least partially formed using the one or more degradable molecules.

37. The drug delivery composition of claim 35, wherein the one or more degradable molecules are attached to the matrix material.

38. The drug delivery composition of claim 35, wherein the matrix material comprises particles formed from the matrix material and wherein the one or more degradable molecules are attached to a surface of a particle formed from the matrix material.

39. The drug delivery composition of claim 35, wherein the degradable molecules function as therapeutic agents.

40. A pharmaceutical formulation comprising:
a pharmaceutically acceptable carrier;
a matrix material; and
one or more degradable molecules of claim 1.

41. The pharmaceutical formulation of claim 40, wherein the matrix material is at least partially formed using the one or more degradable molecules.

42. The pharmaceutical formulation of claim 40, wherein the matrix material is in the form of a particle.
